(19) United States
(12) Patent Application Publication
(10) Pub. No.: US 2002/0114796 A1
(43) Pub. Date: Aug. 22, 2002

(54) Medicament for topical application

(76) Inventor: Johann Eibl, Wien (AT)

Correspondence Address: BAKER BOTTS L.L.P., 30 ROCKEFELLER PLAZA, NEW YORK, NY, 10112, US

(21) Appl. No.: 09/998,575

(22) Filed: Nov. 16, 2001

Related U.S. Application Data
(63) Continuation of application No. PCT/AT00/00141, filed on May 19, 2000.

(30) Foreign Application Priority Data
May 19, 1999 (AT) A89599

(51) Int. Cl. A61K 38/48; A61K 38/53

(52) U.S. Cl. 424/94.1; 424/94.64; 424/94.5

(57) ABSTRACT

The invention relates to a medicament for topical application for the purpose of stopping bleeding and/or closing wounds and/or promoting wound healing, which as active substances —produced conventionally of allogenic plasma or tissue or recombinantly—contains fibrinogen or fibrin, thrombin and one or several tranglutaminase(s), wherein the medicament, as a further active substance, contains one or several protease inhibitor(s) selected from the group consisting of serpins that do not have inhibiting effects on collagenases and elastases, all of the active substances being of allogenic origin and having been subjected to a process for virus depletion and/or virus inactivation, with the proviso that the virus inactivtion of the one or several protease inhibitor(s) has not been carried out in the presence of the other active substances.

The closure of a wound and the related hemostasis are effected physiologically by extravasating blood coagulating in the wound bed, thereby causing the closure of small blood vessels and capillaries. The wound healing setting in subsequently is effected by the aid of the provisional extracellular matrix (ECM) formed by the coagulated blood (Clark, R. A. F. et al., 1982, J. Invest. Dermatol. 70:264-269; Clark, R. A. F. (ed.), 1996, The Molecular and Cellular Biology of Wound Repair, Plenum Press, New York). That matrix, in addition to blood cells, essentially consists of fibrin as the structural substance serving as a reservoir for a number of plasma proteins that are important for the beginning of wound healing, such as fibronectin (Mosesson, M. W. and Umfleet, R., 1970, J. Biol. Chem. 245:5726-5736; Clark, R. A. F. et al., 1982, J. Invest. Dermatol. 70:264-269), vitronectin (Preissner, K. T. and Jenne, D., 1991, Thromb. Haemost. 66:189-194), plasminogen (Castellino, F. J. et al., 1983, Ann. N.Y. Acad. Sci. 408:595-601), plasminogen activator (Thorsen, S. et al., 1972, Thromb. Pathol. Haemost. 28:65-74), plasminogen activator inhibitor (Wagner, O. F. et al., 1989, Blood 70:1645-1653), and alpha₂-plasmin inhibitor (Sakata, Y. and Aoki, N., 1980, J. Clin. Invest. 65:290-297).

The amount of plasminogen activator, plasminogen activator inhibitor and alpha₂-plasmin inhibitor and their mutual ratios exert an essential control on the subsquent degradation process of fibrin. Yet, also other substances such as, e.g., thrombin, TGF-beta and PDGF are contained in that fibrin sceleton, which are necessary for the immigration of cells and the multiple remodeling (reconstruction) of the provisional ECM to the final ECM comprising the appropriate cell populations.

In the first place, granulocytes immigrate into the wound area and into the wound closure in major quantities. They release various substances important for the wound healing procedure to set in such as, in particular, collagenases and elastases, and extracellularly and intracellularly destroy microorganisms that have entered the wound area and are able to propagate there. While the immigration of granulocytes is nearing its end, monocytes including macrophages are occurring in the wound area to an elevated extent. On the third day, fibroblasts will sprout in the wound area, getting as far as to the surface of the wound closure via fibronectin strands. Followed by the ingrowth of blood capillaries, there will occur a number of cell transformations and remodeling processes, which, in most cases, will result in the complete integrity of the injured tissue (Clark, R. A. F.(ed), 1996, The Molecular and Cellular biology of Wound Repair, Plenum Press, New York).

The first attempts to close wounds by the aid of blood plasma, which date back already 80 to 90 years, were not successful because of the relatively low viscosity as compared to blood, the poor adherence in the wound bed and, in the case of the formation of a clot of coagulated plasma, the high fragility of such a clot.

The use of enriched fibrinogen solutions instead of plasma for the purpose of stopping bleeding and closing wounds in the beginning likewise was unsuccessful, but finally a substantial success could be achieved by raising the fibrinogen concentration of such fibrinogen-containing solutions to more than ten times the fibrinogen level in plasma (Löblich, 1975, unpublished communication).

When converting fibrinogen into fibrin, it may happen that the hemostatically effective fibrin wound closure will be detached by wound bed enzymes after some hours, thereby causing afterbleeding. The detachment of the fibrin wound closure from the wound bed is a substantially more frequent and hence more dangerous procedure than the fibrinolysis of the whole fibrin wound closure.

It was proved already by the first successful applications of highly concentrated fibrinogen solutions (Matras, H. et al., 1972, Wr. Med. Wschr. 122:517-523) and the conversion of fibrinogen to fibrin by thrombin in the wound area that any detachment of the fibrin wound closure and the usually involved afterbleedings could be avoided by means of fibrinolysis inhibitors. Among the low-molecular inhibitors assayed, epsilon-aminocaproic acid and derivatives could be proved effective, yet they had the disadvantage of rapidly diffunding out of the coagulated fibrin and of the wound area, and hence losing their topical efficacy.

The admixture of high-molecular inhibitors such as, e.g., aprotinin (Trasylol) was successful by causing only a slow diffusion of the inhibitor from the wound area, yet with the drawback that this was a bovine and, thus, xenogenic protease inhibitor likely to induce potential allergies and anaphylactic reactions. Recently, objections have been raised against the use of animal material for the parenteral application in men also because of the potential transmissibility of zoonoses.

WO-A-99/11301 has proposed to replace aprotinin with elastase inhibitors or other inhibitors effective against leucocyte proteases. According to the findings of the inventor of the present invention, that proposal involves, however, various disadvantages. It is true that those inhibitors act directly or indirectly by the inhibition of enzymes capable of activating the fibrinolytic system, yet they may disturb the setting in of wound healing after granulocyte immigration into the wound area by strongly inhibiting the proteases released by the granulocytes, such as collagenases and elastases.

Another difficulty in producing pharmaceutical medicaments containing fibrinogen and thrombin as well as an allogenic protease inhibitor and a transglutaminase zymogen is due to the virus inactivation of such preparations, which has been required for quite some time. A large portion of the activity of the protease inhibitor or transglutaminase zymogen respectively contained in the preparation will be lost by virus inactivation in most cases such that the preparations obtained after having carried out the virus inactivation process frequently will exhibit but a low activity of the protease inhibitor or transglutarninase zymogen, respectively. This may result in an insufficient inhibition of fibrinolytic enzymes present in the wound bed and, consequently, in the detachment of the fibrin wound closure from the wound bed.

The present invention has as its object to provide a medicament capable of being applied topically for the purpose of stopping bleedings and/or closing wounds and/or promoting wound healing, wherein the use of a xenogenic protease inhibitor is to be avoided while, nevertheless, ensuring to a sufficient extent the inhibition of fibrinolytic enzymes in the wound bed after application of the medicament such that no detachment of the fibrin wound closure from the wound bed will occur. Furthermore, any disturbance of the setting in of wound healing due to an inhibition of the proteases released by the granulocytes immigrated into the wound area, such as collagenases and elastases, by the medicament is to be largely avoided.

For a medicament comprising as active substances-produced conventionally of allogenic plasma or tissue or recombinantly-fibrinogen or fibrin, thrombin and one or several transglutaminase(s), this object is achieved in that the medicament, as a further active substance, contains one or several protease inhibitor(s) selected from the group consisting of serpins that do not have inhibiting effects on collagenases and elastases, all of the active substances being of allogenic origin and having been subjected to a process for virus depletion and/or virus inactivation, with the proviso that the virus inactivation of the one or several protease inhibitor(s) has not been carried out in the presence of the other active substances.

By using as protease inhibitors serpins that do not have inhibiting effects on collagenases and elastases, the inhibition of the proteases released by the granulocytes immigrated into the wound area is largely avoided such that the setting in of wound healing will not be impeded by the medicament according to the invention.

The present invention is further based on the finding that the inhibitory activity of allogenic protease inhibitors will be preserved to a substantially better degree if the latter are subjected to virus inactivation not within a preparation containing one or several of the other active substances of the medicament, but are virus-inactivated separately from the other active substances. In this manner, it is feasible to prepare medicaments according to the invention which contain virus-inactivated allogenic protease inhibitors having sufficient activity so as to inhibit fibrinolytic enzymes in the wound bed after application of the medicament and preventing the detachment of the fibrin wound closure from the wound bed.

In this context, it is noted that the term “virus inactivation” as used for the purposes of the present invention does not encompass processes merely aimed at virus depletion.

The medicament according to the invention, i.a., contains fibrinogen or fibrin as an active substance. This means that, depending on the form of application, either fibrinogen as such or fibrin formed of fibrinogen by the exposure to thrombin is present in the medicament.

The object of the invention is, thus, a composite medicament which constitutes, or develops, a sterile, virus-safe, slowly absorbable and remodelable allogenic provisional extracellular matrix to be applied topically or only forming locally. Since the active substances of these medicaments are of a high molecular character, thus being potential antigens, only allogenic active substances—based on the species to which the medicament is to be applied—are used for the production of these medicaments.

By the addition of further allogenic virus-safe active substances during its production or application, the allogenic provisional extracellular matrix enables the control of the wound healing, in particular, by said active substances being immobilized to the structural substance by transglutaminases and being capable of exerting their effects in the immobilized state or being released during the absorption and remodeling process.

The allogenic transglutaminases contained in the medicament according to the invention, for instance factor XIIIa, cause the allogenic protease inhibitors to be covalently bound to fibrin, thereby practically preventing any diffusion of the inhibitors from the wound area.

A preferred embodiment of the medicament according to the invention is characterized in that all of the active substances are present as an allogenic provisional extracellular matrix in a single pharmaceutical preparation formulated, for instance, as a gel. The medicament in that form is directly usable for topical application.

Suitably, the active substances also may be present in two or several separate pharmaceutical preparations to be mixed prior to or during application, wherein the mixture obtained may be applied in the liquid form or only after its solidification. The preparations may be available in liquid form or frozen or freeze-dried for thawing or reconstitution prior to mixing.

The pharmaceutical preparations may be mixed, for instance, at least ten minutes prior to their application on a wound surface, whereupon the mixture will solidify by forming a slowly absorbable and remodelable allogenic provisional extracellular matrix, which is applied on the wound area. It is, however, also feasible to mix the pharmaceutical preparations and apply the liquid mixture topically immediately thereupon, for instance in the form of a spray, so that a slowly absorbable, remodelable allogenic provisional extracellular matrix will form only locally.

It is preferred that the concentration of fibrinogen and protease inhibitors in the medicament be chosen such that the liquid mixture contains at least 30 g/l of fibrinogen and 500 arbitrary plasma units of protease inhibitors/1.

Preferably, fibrinogen and thrombin are each present in separate pharmaceutical preparations, the remaining active substances, independent of one another, being contained in one or both of said preparations and/or in a further preparation.

By appropriately composing the thrombin-containing pharmaceutical preparation, the invention enables substantial quantities of thrombin to be still generated in the formed fibrin even some hours after mixing with the fibrinogen-containing solution and solidification of the mixture. This is of great importance both to the stability and to the quality of the allogenic provisional extracellular matrix thus formed.

The pharmaceutical preparations contained in the medicaments according to the invention advantageously also may be applied on allogenic or biocompatible carrier materials subjected to a process for virus depletion and/or virus inactivation. Depending on the purpose of application, the carrier materials may have different forms. The preparation of a ready-made medicament may, for instance, be effected by applying the yet liquid mixture of the pharmaceutical preparations onto the carrier material.

A preferred embodiment of the medicament according to the invention is characterized in that it contains, as further active substances, allogenic collagens subjected to a process for virus depletion and/or virus inactivation. The additional use of collagens in the preparation or formation of the allogenic provisional extracellular matrix consideraby enhances its biomechanical quality.

Suitably, the medicament according to the invention contains one or several further allogenic active substance(s) subjected to a process for virus depletion and/or virus inactivation and selected from the group consisting of fibronectin, vitronectin, thrombospondin, tenascin, laminin and proteoglycans. By adding substances such as, e.g., vitronectin, further enhancement of the effect of certain protease inhibitors may be achieved.

It is, moreover, advantageous if the medicament according to the invention contains one or several further allogenic active substances subjected to a process for virus depletion and/or virus inactivation and selected from the group consisting of growth factors, chemotactic substances, cell stimulating and/or proliferation enhancing enzymes and enzyme inhibitors, proliferation inhibiting enzymes and enzyme inhibitors, cytokines and particulately formed cell elements. The application of allogenic enzyme inhibitors in the context of wound closure offers the opportunity to obtain permanent healing, in particular, in the case of skin ulcera.

Preferably, the medicament according to the invention contains additions of allogenic plasmatic enzymes, or enzymes obtained from tissues, zymogens and/or enzyme inhibitors subjected to a process for virus depletion and/or virus inactivation.

Depending on the clinical requirement, it is suitable if the medicament according to the invention is supplemented with antiadherent, antiphlogistic, antimicrobial and/or cytostatic agents subjected to a process for virus depletion and/or virus inactivation, if necessary. The addition of antiphlogistic and antiadherent allogenic agents such as, e.g., certain immunoglobulins or other active substances occurring in plasma and having such effects renders feasible the application of an allogenic provisional extracellular matrix even where postoperative adhesions must be feared.

The allogenic provisional extracellular matrix forming in the application of the medicament or being present as such may function as a reservoir or depot for substances that are to be slowly released from the matrix. A preferred embodiment of the medicament according to the invention, therefore, is characterized in that it contains additions of active substances that are to be absorbed slowly and, if necessary, have been subjected to a process for virus depletion and/or virus inactivation.

Advantageously, allogenic zymogens and/or enzymes of the coagulation cascade, which have been subjected to a process for virus depletion and/or virus inactivation are additionally contained in the thrombin-containing pharmaceutical preparation of the medicament according to the invention.

Preferably, the medicament according to the invention additionally contains allogenic particulate cell elements, cells and/or tissues, which may be applied on virus-safe microspheres consisting of allogenic fibrin and/or allogenic collagen and/or allogenic collagen plus allogenic fibrin.

A drawback of the hitherto known fibrinogen- or fibrin-containing pharmaceutical preparations is the relatively high fragility of the fibrin formed, even if very high percentage fibrinogen solutions are used.

Although the fibrin forming during coagulation is crosslinked to form a sceleton on account of the presence of factor XIIIa-a transglutaminase forming in the pharmaceutical preparation of factor XIII contained in the same by thrombin action—, yet high thrombin and factor XIII concentrations must still be contained, or form, in the fibrin in order to ensure as complete a reaction as possible to occur between the crosslinking sites within the fibrin. Even such fibrins are, however, difficult to use because of the persistently present fragility and the locally formed fibrins likewise continue to exhibit poor mechanical strengths.

A preferred embodiment of the medicament according to the invention, therefore, is characterized in that the allogenic provisional extracellular matrix is solidified by the application of pressure and/or by means of dehydrating agents. In that case, the matrix may be treated with allogenic transglutaminases prior to, during and/or after solidification by dehydration.

Such allogenic matrices, due to their sufficient mechanical stability, may also be used as artificial allogenic skins. It has, thus, also become feasible to produce a skin replacement exclusively of human substances and additionally introduce in such a preparation also particulate cell elements, cells and tissues, thus offering new opportunities of medical use.

It is also feasible by means of the pharmaceutical preparations according to the invention to fill up body sites afflicted by a strong degradation of endogenous tissue so as to obtain long-term healing.

Concentrated fibrinogen solutions involve a number of drawbacks. They offer reduced storage stability and must be deepfrozen or freeze-dried for storage and cannot always be made usable or reconstituted in a satisfactory manner by thawing or redissolving. Moreover, the dissolution of a fibrinogen lyophilisate requires some time. Solubilizers or readily soluble fibrinogens, in most cases, are cytotoxic and, therefore, not suitable for an undisturbed wound healing.

The greatest difficulty in the storage of fibrinogen-containing products is the instability of fibrinogen, since traces of impurities of coagulation factors will cause the slow conversion of fibrinogen to insoluble fibrin, which no longer will allow the application of such a pharmaceutical preparation.

The present invention, consequently, also relates to a process for preparing a fibrinogen-containing solution which, as such or as a component of the medicaments according to the invention, is storable at refrigerator temperature or room temperature, wherein the fibrinogen solution or a fibrinogen-fibronectin solution is prepared of recombinantly produced fibrinogen or of fibrinogen obtained from plasma by fractionation with glycine at temperatures of below 0° C.

Fibrinogen-containing solutions prepared in accordance with the invention are stable and may be stored in the liquid state with or without stabilizers for more than two years at a temperature of 4°-8° C. or in a frozen or lyophilized state without insoluble fibrin precipitating or fibrinogen cleavage products forming due to the formation of plasmin during storage to an extent disturbing coagulability. Such solutions will coagulate neither after the addition of thromboplastin and taipan viper venom nor by the addition of activated partial thrombo-plastin. No coagulation procedures will be induced by storage in the deepfrozen state and after thawing of the fibrinogen-containing solutions according to the invention, and the solutions obtained will be stable for at least some hours. Lyophilized preparations are readily and completely reconstitutable by appropriate solvents.

The present invention also provides for a medicament containing a highly purified fibrinogen-containing or fibrinogen-fibronectin-containing preparation whose aPTT and taipan viper venom prothrombin time at 37° C. are no less than 200 or 300 seconds, respectively, and which has a stability enabling it to be stored for more than two years at a temperature of 4°-8° C. in the liquid state with or without stabilizers as a component of medicaments or as such or in a frozen or lyophilized state, without causing insoluble fibrin to precipitate or fibrinogen cleavage products to form due to the formation of plasmin during storage.

Despite the use of exclusively allogenic active substances, neoantigens may form in the virus inactivation of medicaments composed of allogenic active substances, either by the interaction of two or several active substances of the medicament during virus inactivation and/or by the interaction with impurities still contained in the active substances.

Another drawback in the virus inactivation of mixtures of active substances resides in the frequently strongly different inactivation of the individual active substances themselves, thereby rendering difficult or impossible the formulation of the respective medicaments.

The invention, therefore, also relates to a process for obtaining pathogen-free active substances contained in the medicaments according to the invention, comprising a combination of depletion processes and inactivation processes including steps of virus depletion and virus inactivation, wherein ultracentrifugations and ultrafiltrations including nanofiltrations and/or adsorptions of pathogens at temperatures of below 0° C. are used for virus depletion and at least two different virus inactivation processes are carried out, in which heat pulse processes of below 3 seconds and/or intensive laser pulse radiation with or without photodynamic substances and/or detergents are used along with hydrophobic wetting agents.

The process according to the invention enables the virus inactivation of active substances having sufficient degrees of purity prior to their formulation, whereby it is ensured by an appropriate choice of the inactivation processes that all of the active substances are equally virus inactivated without excessive losses. In that manner, the induction or triggering of allergies, anaphylactic reactions and autoimmune processes by medicaments containing such active substances may be avoided to the major extent.

The process according to the invention, in particular, also enables a particularly gentle virus inactivation of the protease inhibitors contained in the medicaments according to the invention.

The invention, furthermore, relates to a process for covalently binding to a biological matrix active substances contained in the medicaments according to the invention, wherein high concentrations of allogenic virus-depleted and/or virus-inactivated transglutaminases, which exceed the 10 time concentration range of the activated concentration of the factor XIIIa zymogen occurring in plasma, are used for the enzymatic catalysis of the binding reaction.

Due to a sufficiently high and sustained thrombin concentration in the allogenic provisional extracellular matrix and with a sufficient quantity of factor XIII being present, a sufficient quantity of factor XIIIa can be formed, which, on the one hand, causes crosslinking of the basic substance(s) fibrin with or without collagen and, on the other hand, generates TAFI in the presence of the TAFI zymogen. TAFI cleaves the plasmin receptor from the fibrin, thereby providing a high stability of the fibrin-containing allogenic provisional extracellular matrix against fibrinolytic enzymes. Furthermore, it is feasible according to the invention, due to a high and sustained transglutaminase concentration in the allogenic provisional extracellular matrix, to covalently bind appropriately virus-inactivated allogenic protease inhibitors such as alpha₂-antiplasmin, PAI-1 and others, thus preventing them from diffusing out of the allogenic provisional extracellular matrix. The same holds for other active substances having protein character and capable of being covalently bound to the sceleton substance by the aid of transglutaminases.

The invention, furthermore, relates to a process for preparing a fibrin-containing low-water gel having a water content of between 20 and 90%, wherein water is removed from the fibrin-containing gel by applying atoxic, pharmaceutically usable dehydrating agents, in particular polyethylene glycol, or by introducing the gel into such agents, wherein the gel may be treated with transglutaminases before or after dehydration.

The production of a fibrin-containing gel poor in water, having a water content of between 20 and 90%, in accordance with the invention also may be effected by a process in which water is removed from the fibrin-containing gel by applying high pressures, said pressures being gradually increased in order to avoid destruction of the gel. The gel may be treated with transglutaminases before, during and/or after the application of pressure.

The invention also relates to a process for solidifying a fibrin-containing gel with and without dehydration, characterized in that the fibrin-containing gel is placed in one or several metallic ion-containing solution(s), in particular solutions containing zinc and aluminum ions in concentrations ranging from 0.01 to 2 molar.

Furthermore, the present invention provides a lyophilized fibrin-containing gel prepared by the addition of plasticizers, in particular glycol, prior to its solidification and lyophilization.

The aforementioned processes according to the invention for preparing fibrin-containing low-water gels as well as solidifying fibrin-containing gels with a particular advantage may be used for producing medicaments according to the invention.

A disadvantage of the hitherto used fibrinogen-containing preparations in the topical application of fibrinogen-containing mixtures of active substances is their too low viscosity prior to solidification. As a result, the applied mixture may readily and quickly run off the site of application, which calls for a higher consumption of the medicament, on the one hand, and leads to an unsatisfactory application, in particular in connection with surgical interventions where an application is required in the operation area, on the other hand.

The invention, therefore, also relates to a process for preparing a highly viscous fibrinogen-containing solution, wherein a sterile, virus-depleted and/or virus-inactivated fibrinogen solution per gram of fibrinogen is mixed slowly and under vigorous stirring and under sterile conditions with a one hundredth to one tenth unit of sterile, virus-depleted and/or virus-inactivated thrombin dissolved in a volume as small as possible. The highly viscous fibrinogen-containing solutions prepared by the process according to the invention with particular advantage may be used for preparing medicaments according to the invention.

The present invention, furthermore, provides a process for determining the adherence of a fibrin clot in the wound bed, in which fibrin clots at increasing amounts of protease inhibitors are formed on the surfaces of suitable tissue cultures, in particular human fibroblasts, with a small addition of one or several staining substances and/or one or several water-insoluble substances yielding a stained or opalescent fibrin clot, and the tissue cultures are kept in motion by slight shaking or rotating while determining the time after which the fibrin clots detach from the surface of the tissue culture.

The process according to the invention enables the determination of those amounts of protease inhibitors which are required in an allogenic provisional extracellular matrix as, for instance, present in, or formed by, the medicaments according to the invention in order to leave such a matrix on the site of application for at least several days and, thus, prevent its detachment from the wound bed as well as afterbleeding caused thereby.

### EXAMPLES

### Example 1

Preparation of a Stable Fibrinogen Solution

1001 plasma suitable for the preparation of medicaments, or the Cohn fraction I obtained therefrom by means of a cold ethanol precipitation, or cryoprecipitate obtained by freezing the plasma and careful thawing are used as a starting material.

The plasma as such, the Cohn fraction I and the cryoprecipitate after dissolution in about 20 liters of 0.9% NaCl and 0.1% sodium citrate buffer pH 7, are supplemented to saturation with solid glycine having a degree of purity suitable for pharmaceutical preparations while being cooled to −2° C. to −3° C., stored at that temperature for a minimum of 10 to a maximum of 15 hours, separated from undissolved glycine, and the fibrinogen-fibronectin-containing precipitate is separated by centrifugation in a high-speed centrifuge.

The supernatants may be used for further processing in order to isolate other plasma proteins therefrom.

The gelatinous precipitate is removed from the rotor of the centrifuge, dissolved and precipitated with glycine as before. The sediment obtained after centrifugation is dissolved in 0.1% citrate and this procedure of reprecipitation is repeated until a sample of the obtained sediment taken up in distilled water at a fibrinogen content of 0.1% to 0.2% at 37° C. with a PTT reagent yields an aPTT of no less than 200 s. Likewise, a coagulation time of no less than 300 s must be reached after the addition of thromboplastin and taipan viper venom.

The redissolved final precipitate suitable for further processing is largely freed of glycine by diafiltration against an 0.1% citrate solution and thereby concentrated to a protein content of from 2% to 3%.

The thus obtained fibrinogen still contains a substantial amount of fibronectin. If desired, fibronectin may be separated by precipitation with 17% glycine at −2° C. to −3° C. In doing so, practically all of the fibrinogen is precipitated and fibronectin with small amounts of fibrinogen is contained in the supernatant. By saturation with glycine, the two proteins may be precipitated and recovered together. Possibly present low activities of plasminogen activator or plasmin may be rendered ineffective by the addition of small quantities of low-molecular inhibitors such as epsilon-aminocaproic acid or derivatives.

The virus inactivation of the highly purified fibrinogen or of the fibrinogen-fibronectin complex may be effected by admixing the plasma of the dissolved Cohn fraction I or of the dissolved cryoprecipitate with detergents and wetting agents or by way of a heat pulse or laser light process. The virus-inactivated fibrinogen solution may be stored as such at a refrigerator temperature at 4° C. to 8° C., or may be deepfrozen or lyophilized for storage.

### Example 2

Preparation of a thrombin solution having thrombin-generating potential

This thrombin solution is obtained by mixing equivalent amounts of two solutions obtained from human plasma suitable for the production of medicaments for use in humans. The two solutions are free of pyrogen, free of Ca ions and sterile. One solution contains virus-safe thrombin in a concentration to be selected from 20 to 2,500 units/ml, the other solution contains a virus-safe prothrombin coagulation factor mixture capable of generating at least 1,000 thrombin units per ml after the addition of Ca ions.

To that mixture is admixed a one hundredth volume part of a sterile solution of 10% polyethylene glycol (pharmaceutical purity) and, depending on the dosage desired, the mixture is then filled into vials or ready-made syringes and deepfrozen, optionally freeze-dried, sealed, stored, labeled and packed.

### Example 3

Production of medicaments for topical application and suitable for use in humans, from fibrin-containing and thrombin-containing pharmaceutical preparations.

800 ml of a pyrogen-free liquid active substance preparation according to Example 1 obtained from human plasma suitable for the production of medicaments for use in humans and having a fibrinogen content of at least 6% are used to dissolve a freeze-dried, sterile, pyrogen-free virus-inactivated pharmaceutical preparation obtained from plasma suitable for the production of medicaments for use in humans and containing 1,200 arbitrary units, based on human plasma, of PAI-1 or any other serpin or serpin mixture that does not exhibit a collagenase- or elastase-inhibiting effect and a transglutaminase zymogen content equivalent to at least 4,000 units of factor XIII. In addition, the preparation contains 2 g CaCl₂.

This fibrinogen-containing pharmaceutical preparation is then mixed with the thrombin-containing preparation indicated in Example 2 after reconstitution of the same with a suitable amount of water for injection, i.e., 4 parts of the fibrinogen-containing preparation are mixed with 1 part of the .thrombin-containing preparation.

### Example 4

Preparation of an allogenic provisional extracellular matrix as a medicament.

A defined amount of a mixture according to Example 3 is poured into a desired sterilized mold and incubated for 5 hours under sterile conditions at between 35° C. and 37° C. The sterile pyrogen-free, virus-safe extracellular matrix formed in the mold, which is allogenic for use in men, is packed into sterile water-vapor-tight polyethylene sheaths, sealed and stored at refrigerator temperature at 4° C. to 8° C. to be released according to the respective quality controls. After labeling, packing and release, the medicament may be put on the market.

### Example 5

Preparation of an allogenic provisional extracellular matrix by mixing three pharmaceutical preparations offered as a composite medicament.

The medicament contains three pharmaceutical preparations which, after mixing, form an allogenic provisional extracellular matrix:

a. a 6% fibrinogen solution according to Example 3,

b. a freeze-dried mixture of transglutaminase zymogen, serpin or serpin mixture, free of collagenase and elastase inhibiting effects and calcium chloride in the amounts indicated in Example 3

c. a freeze-dried thrombin solution according to Example 2.

In addition, the medicament contains water for injection, which is used in order to dissolve the thrombin-containing pharmaceutical preparation according to Example 2, while the pharmaceutical preparation containing the transglutaminase zymogen and the serpin is dissolved in the 6% fibrinogen solution according to Example 3. After mixing of the fibrinogen-containing solution with that containing thrombin and prior to the coagulation of that mixture, the latter either is introduced into a desired sterile pyrogen-free mold and removed from the mold after solidification and applied topically or the still liquid mixture is applied topically to the desired site or sites with a provisional extracellular matrix allogenic to man locally forming only then.

### Example 6

Biomechanically reinforced allogenic provisional extracellular matrix using allogenic collagen.

Into the mixture of the pharmaceutical preparations according to Examples 4 and 5 may also be introduced fibrillary sterile, pyrogen-free, virus-safe human collagen of between 5 and 100 mg/ml, or the mixture is applied on a rapidly absorbing foamed collagen fleece with the thrombin content of the mixture being adjusted such that fibrin formation occurs only after the end of the absorption procedure. Such a fleece may be used immediately after the setting in of the coagulation procedure or worked up to a ready-made medicament packed under sterile conditions after a 5 hour incubation at between 35° C. and 37° C.

### Example 7

Determination of the required concentrations of non-collagenase-inhibiting or non-elastase-inhibiting serpins.

A selected fibroblast cell line whose selection has been effected in terms of maximum fibrinolytic activity is introduced at a cellular density of about 10⁷ cells per ml into tissue culture flasks each having a growth area of at least 30 cm² in such an amount that 10⁵ cells are present per cm² tissue culture area. After the addition of at least 10 ml tissue culture medium, the flasks are incubated until a complete cell lawn has formed. If required, a change of medium is effected at predetermined intervals.

After a complete cell lawn has grown, the medium is removed and the cell lawn is washed twice with at least 20 ml medium each, the medium is removed from the flask to the major extent and 50 μl of a mixture set out below are each applied, by means of suitable pipettes, onto the exactly horizontally put cell lawn on sites marked on the external sides of the flasks and, after this, the tissue culture flasks are closed and stored for 1 hour at room temperature, then are again filled with medium as before, closed and kept in an incubator at 37° C. on a tissue culture rocker at a rocking rate of about 10 tilts per min and then inspected for the detachment of the fibrin-containing clots applied on the cell lawn after 30, 100 and 300 minutes and, thereafter, every 8 hours.

The application of the mixtures indicated below is effected in the following manner:

Plasma supplemented prior to mixing with methylene blue to the desired extent and one thousand units of thrombin/ml plasma is applied onto six previously marked sites. By way of experiment, mixtures according to Example 3 are prepared, which are stained with a desired amount of phenolic red and contain different amounts of plasmatic serpins or recombinantly produced serpins. Of each serpin concentration, 6×50 μl of the mixture are likewise applied on the marked sites. After solidification of the mixture samples applied, it is proceeded as indicated above.

The points of time at which the fibrin-containing samples detach from the tissue culture surface are then determined. The detachment times of the serpin-containing samples are compared to those of the plasma samples. Advantageously, those serpin concentrations are chosen, which induce detachment times at least equal to those observed with the plasma-produced clots applied.

What is claimed is:

1. A topical medicament intended for stopping bleeding, closing a wound, or promoting wound healing in a subject in need of such treatment, comprising the following active agents in therapeutic amounts:
(i) an agent selected from the group consisting of fibrinogen and fibrin;
(ii) thrombin;
(iii) a transglutaminase; and
(iv) a serpin protease inhibitor which does not inhibit collagenase and elastase; wherein the active agents may be obtained from a source selected from the group of allogenic plasma, allogenic tissue, and recombinant production; and wherein an active substance of allogenic origin is subjected to a process selected from the group consisting of virus depletion, virus inactivation and a combination thereof; provided that where such a process is applied to the serpin protease inhibitor, it is not applied in the presence of one or more of the other active agents.

2. The medicament of claim 1, further comprising allogenic collagens subjected to a process selected from the group consisting of virus depletion, virus inactivation, and a combination thereof.

3. The medicament of claim 1, further comprising one or more allogenic active agent selected from the group consisting of fibronectin, vitronectin, thrombospondin, tenascin, laminin and proteoglycans, wherein the allogenic active agent is subjected to a process selected from the group consisting of virus depletion, virus inactivation, and a combination thereof.

4. The medicament of claim 2, further comprising one or more allogenic active agent selected from the group consisting of fibronectin, vitronectin, thrombospondin, tenascin, laminin and proteoglycans, wherein the allogenic active agent is subjected to a process selected from the group consisting of virus depletion, virus inactivation, and a combination thereof.

5. The medicament of claim 1, further comprising one or more allogenic active agent selected from the group consisting of growth factors, chemotactic substancs, cell stimulating enzymes, cell proliferation enhancing enzymes, inhibitors of cell stimulating enzymes, inhibitors of cell proliferation enhancing enzymes, cell proliferation inhibiting enzymes, inhibitors of cell proliferation inhibiting enzymes, cytokines, and particulately formed cell elements, wherein the allogenic active agent is subjected to a process selected from the group consisting of virus depletion, virus inactivation, and a combination thereof.

6. The medicament of claim 2, further comprising one or more allogenic active agent selected from the group consisting of growth factors, chemotactic substancs, cell stimulating enzymes, cell proliferation enhancing enzymes, inhibitors of cell stimulating enzymes, inhibitors of cell proliferation enhancing enzymes, cell proliferation inhibiting enzymes, inhibitors of cell proliferation inhibiting enzymes, cytokines, and particulately formed cell elements, wherein the allogenic active agent is subjected to a process selected from the group consisting of virus depletion, virus inactivation, and a combination thereof.

7. The medicament of claim 3, further comprising one or more allogenic active agent selected from the group consisting of growth factors, chemotactic substancs, cell stimulating enzymes, cell proliferation enhancing enzymes, inhibitors of cell stimulating enzymes, inhibitors of cell proliferation enhancing enzymes, cell proliferation inhibiting enzymes, inhibitors of cell proliferation inhibiting enzymes, cytokines, and particulately formed cell elements, wherein the allogenic active agent is subjected to a process selected from the group consisting of virus depletion, virus inactivation, and a combination thereof.

8. The medicament of claim 4, further comprising one or more allogenic active agent selected from the group consisting of growth factors, chemotactic substancs, cell stimulating enzymes, cell proliferation enhancing enzymes, inhibitors of cell stimulating enzymes, inhibitors of cell proliferation enhancing enzymes, cell proliferation inhibiting enzymes, inhibitors of cell proliferation inhibiting enzymes, cytokines, and particulately formed cell elements, wherein the allogenic active agent is subjected to a process selected from the group consisting of virus depletion, virus inactivation, and a combination thereof.

9. The medicament of claim 1, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

10. The medicament of claim 2, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

11. The medicament of claim 3, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

12. The medicament of claim 4, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

13. The medicament of claim 5, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

14. The medicament of claim 6, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

15. The medicament of claim 7, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

16. The medicament of claim 8, further comprising one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

17. The medicament of claim 1, further comprising one or more active agent selected from the group consisting of antiadherent agents, antiphlogistic agents, antimicrobial agents, cytostatic agents, active substances that are absorbed slowly, and combinations thereof.

18. The medicament of claim 2, further comprising one or more active agent selected from the group consisting of anti adherent agents, antiphlogistic agents, antimicrobial agents, cytostatic agents, active substances that are absorbed slowly, and combinations thereof.

19. The medicament of claim 3, further comprising one or more active agent selected from the group consisting of antiadherent agents, antiphlogistic agents, antimicrobial agents, cytostatic agents, active substances that are absorbed slowly, and combinations thereof.

20. The medicament of claim 4, further comprising one or more active agent selected from the group consisting of antiadherent agents, antiphlogistic agents, antimicrobial agents, cytostatic agents, active substances that are absorbed slowly, and combinations thereof.

21. The medicament of claim 5, further comprising one or more active agent selected from the group consisting of antiadherent agents, antiphlogistic agents, antimicrobial agents, cytostatic agents, active substances that are absorbed slowly, and combinations thereof.

22. The medicament of claim 6, further comprising one or more active agent selected from the group consisting of antiadherent agents, antiphlogistic agents, antimicrobial agents, cytostatic agents, active substances that are absorbed slowly, and combinations thereof.

23. The medicament of claim 7, further comprising one or more active agent selected from the group consisting of antiadherent agents, antiphlogistic agents, antimicrobial agents, cytostatic agents, active substances that are absorbed slowly, and combinations thereof.

24. The medicament of claim 8, further comprising one or more active agent selected from the group consisting of antiadherent agents, antiphlogistic agents, antimicrobial agents, cytostatic agents, active substances that are absorbed slowly, and combinations thereof.

25. The medicament of claim 9, further comprising one or more active agent selected from the group consisting of antiadherent agents, antiphlogistic agents, antimicrobial agents, cytostatic agents, active substances that are absorbed slowly, and combinations thereof.

26. The medicament of claim 10, further comprising one or more active agent selected from the group consisting of antiadherent agents, antiphlogistic agents, antimicrobial agents, cytostatic agents, active substances that are absorbed slowly, and combinations thereof.

27. The medicament of claim 11, further comprising one or more active agent selected from the group consisting of antiadherent agents, antiphlogistic agents, antimicrobial agents, cytostatic agents, active substances that are absorbed slowly, and combinations thereof.

28. The medicament of claim 12, further comprising one or more active agent selected from the group consisting of antiadherent agents, antiphlogistic agents, antimicrobial agents, cytostatic agents, active substances that are absorbed slowly, and combinations thereof.

29. The medicament of claim 13, further comprising one or more active agent selected from the group consisting of antiadherent agents, antiphlogistic agents, antimicrobial agents, cytostatic agents, active substances that are absorbed slowly, and combinations thereof.

30. The medicament of claim 14, further comprising one or more active agent selected from the group consisting of anti adherent agents, antiphlogistic agents, antimicrobial agents, cytostatic agents, active substances that are absorbed slowly, and combinations thereof.

31. The medicament of claim 15, further comprising one or more active agent selected from the group consisting of antiadherent agents, antiphlogistic agents, antimicrobial agents, cytostatic agents, active substances that are absorbed slowly, and combinations thereof.

32. The medicament of claim 16, further comprising one or more active agent selected from the group consisting of antiadherent agents, antiphlogistic agents, antimicrobial agents, cytostatic agents, active substances that are absorbed slowly, and combinations thereof.

33. The medicament of claim 1, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

34. The medicament of claim 2, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

35. The medicament of claim 3, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

36. The medicament of claim 4, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

37. The medicament of claim 5, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

38. The medicament of claim 6, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

39. The medicament of claim 7, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

40. The medicament of claim 8, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

41. The medicament of claim 9, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

42. The medicament of claim 10, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

43. The medicament of claim 11, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

44. The medicament of claim 12, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

45. The medicament of claim 13, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

46. The medicament of claim 14, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

47. The medicament of claim 15, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

48. The medicament of claim 16, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

49. The medicament of claim 17, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

50. The medicament of claim 18, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

51. The medicament of claim 19, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

52. The medicament of claim 20, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

53. The medicament of claim 21, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

54. The medicament of claim 22, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

55. The medicament of claim 23, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

56. The medicament of claim 24, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

57. The medicament of claim 25, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

58. The medicament of claim 26, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

59. The medicament of claim 27, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

60. The medicament of claim 28, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

61. The medicament of claim 29, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

62. The medicament of claim 30, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

63. The medicament of claim 31, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

64. The medicament of claim 32, further comprising one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

65. The medicament of claim 1, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

66. The medicament of claim 2, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

67. The medicament of claim 5, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

68. The medicament of claim 6, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

69. The medicament of claim 9, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

70. The medicament of claim 10, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

71. The medicament of claim 13, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

72. The medicament of claim 14, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

73. The medicament of claim 17, further comprising an allogenic component-selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

74. The medicament of claim 18, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

75. The medicament of claim 21, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

76. The medicament of claim 22, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

77. The medicament of claim 25, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

78. The medicament of claim 26, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

79. The medicament of claim 29, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

80. The medicament of claim 30, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

81. The medicament of claim 31, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

82. The medicament of claim 33, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

83. The medicament of claim 34, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

84. The medicament of claim 37, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

85. The medicament of claim 38, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

86. The medicament of claim 41, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

87. The medicament of claim 42, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

88. The medicament of claim 45, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

89. The medicament of claim 46, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

90. The medicament of claim 49, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

91. The medicament of claim 50, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

92. The medicament of claim 53, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

93. The medicament of claim 54, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

94. The medicament of claim 57, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

95. The medicament of claim 58, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

96. The medicament of claim 61, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

97. The medicament of claim 62, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

98. The medicament of claim 63, further comprising an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.

99. The medicament of claim 1, wherein all of the active substances are present as an allogenic provisional extracellular matrix in a single pharmaceutical preparation.

100. The medicament of claim 1, wherein the active substances are present in separate pharmaceutical preparations to be mixed prior to or during application, wherein the mixture obtained may be applied in a form selected from the group consisting of liquid form and solid form.

101. The medicament of claim 100, wherein fibrinogen and thrombin are each present in a separate pharmaceutical preparation and the remaining active substances, independent of one another, are contained in either the fibrinogen containing preparation, the thrombin-containing preparation, or another pharmaceutical preparation.

102. The medicament of claim 1, wherein one or more of the active agents are applied on carrier materials subjected to a process selected from the group of virus depletion, virus inactivation, and a combination thereof.

103. The medicament of claim 1, wherein the medicament is solidified into an allogenic provisional extracellular matrix by a means selected from the group consisting of the application of pressure and the use of dehydrating agents.

104. The medicament of claim 103, wherein the allogenic provisional matrix is solidified by the use of dehydrating agents and treated with allogenic transglutaminases.

105. A process for preparing a fibrinogen-containing solution which is storable at refrigerator temperature or room temperature, comprising a step selected from the group consisting of preparing fibrinogen recombinantly and purifying fibrinogen from plasma by fractionation with glycine at a temperature equal to or less than zero degrees Celsius.

106. The process of claim 105, wherein the solution further comprises fibronectin.

107. A medicament comprising a highly purified fibrinogen-containing preparation having (i) an aPTT, measured at 37° C., of at least 200 seconds; (ii) a taipan viper venom prothrombin time, measured at 37° C., of at least 300 seconds; and (iii) a stability that permits its storage for more than two years at a temperature of 4°-8° C. without the formation of an insoluble fibrin precipitate or the formation of fibrinogen cleavage products.

108. A medicament comprising a highly purified fibrinogen-fibronectin-fibronectin-containing preparation having (i) an aPTT, measured at 37° C, of at least 200 seconds; (ii) a taipan viper venom prothrombin time, measured at 37° C., of at least 300 seconds; and (iii) a stability that permits its storage for more than two years at a temperature of 4°-8° C. without the formation of an insoluble fibrin precipitate or the formation of fibrinogen cleavage products.

109. A process for obtaining a pathogen-free active substance comprising (i) depleting virus in the substance by a process selected from the group consisting of ultracentrifugation, ultrafiltration, nanofiltration, adsorption, and combinations thereof, where the process is carried out a temperature below zero degrees Celsius; and (ii) inactivating virus in the substance by subjecting the substance to at least two of the processes in the group consisting of heat pulse processes carried out for less than three seconds, intensive laser pulse radiation, and exposure to detergents in the presence of hydrophobic wetting agents.

110. A process for covalently binding an active agent to a biological matrix comprising catalyzing the binding reaction with an allogenic transglutaminase at a concentration more than ten-fold greater than the concentration of activated factor XIIIa zymogen in plasma, wherein the transglutaminase has been treated with a process selected from the group consisting of virus depletion, virus inactivation, and a combination thereof.

111. A process for preparing a fibrin-containing gel having a water content between 20 and 90 percent, comprising preparing a fibrin containing gel and then exposing the gel to an atoxic, pharmaceutically suitable dehydrating agent.

112. The process of claim 111, wherein the dehydrating agent is polyethylene glycol.

113. A process for preparing a fibrin-containing gel having a water content of between 20 and 90 percent, comprising preparing a fibrin-containing gel and then removing water from the gel by applying pressure, wherein the pressure is increased gradually to avoid destroying the gel.

114. The process of claim 111, further comprising the step of exposing the gel to a transglutaminase.

115. The process of claim 112, further comprising the step of exposing the gel to a transglutaminase.

114. The process of claim 113, further comprising the step of exposing the gel to a transglutaminase.

115. A process for solidifying a fibrin-containing gel, comprising preparing a fibrin-containing gel and then placing the gel in a solution containing metallic ions.

116. The process of claim 115 wherein the solution contains zinc and aluminum ions at concentrations between 0.01 to 2 molar.

117. A lyophilized fibrin-containing gel prepared by a process comprising the steps of adding a plasticizer to the gel prior to its solidification and lyophilization.

118. The gel of claim 117, wherein the plasticizer is glycol.

119. A process for preparing a highly viscous fibrinogen-containing solution, comprising the step of mixing (a) a fibrinogen solution which has been subjected to a process selected from the group consisting of virus depletion, virus inactivation, and a combination thereof with (b) a thrombin solution which has been subjected to a process selected from the group consisting of virus depletion, virus inactivation, and a combination thereof, wherein the relative amounts of fibrinogen to thrombin are one gram of fibrinogen to between b 0.01 and 0.1 units of thrombin.

120. A process for determining the adherence of a fibrin clot in a wound bed, comprising the steps of (i) forming a fibrin clot using increasing amounts of protease inhibitors on the surface of a tissue culture; (ii) rendering the clot detectable by exposing the clot to a detection agent selected from the group consisting of staining substances and water-insoluble substances; (iii) subjecting the tissue culture to a shaking or rotating motion; and (iv) determining the time after which the fibrin clot detaches from the surface of the tissue culture.

121. A method of treating a wound of a subject, comprising applying, to the wound, a topical medicament comprising the following active agents in therapeutic amounts:
(i) an agent selected from the group consisting of fibrinogen and fibrin;
(ii) thrombin;
(iii) a transglutaminase; and
(iv) a serpin protease inhibitor which does not inhibit collagenase and elastase; wherein the active agents may be obtained from a source selected from the group of allogenic plasma, allogenic tissue, and recombinant production; and wherein an active substance of allogenic origin is subjected to a process selected from the group consisting of virus depletion, virus inactivation and a combination thereof; provided that where such a process is applied to the serpin protease inhibitor, it is not applied in the presence of one or more of the other active agents.

122. The method of claim 121, wherein the medicament further comprises allogenic collagens subjected to a process selected from the group consisting of virus depletion, virus inactivation, and a combination thereof.

123. The method of claim 121, wherein the medicament further comprises one or more allogenic active agent selected from the group consisting of fibronectin, vitronectin, thrombospondin, tenascin, laminin and proteoglycans, wherein the allogenic active agent is subjected to a process selected from the group consisting of virus depletion, virus inactivation, and a combination thereof.

124. The method of claim 121, wherein the medicament further comprises one or more allogenic active agent selected from the group consisting of growth factors, chemotactic substancs, cell stimulating enzymes, cell proliferation enhancing enzymes, inhibitors of cell stimulating enzymes, inhibitors of cell proliferation enhancing enzymes, cell proliferation inhibiting enzymes, inhibitors of cell proliferation inhibiting enzymes, cytokines, and particulately formed cell elements, wherein the allogenic active agent is subjected to a process selected from the group consisting of virus depletion, virus inactivation, and a combination thereof.

125. The method of claim 121, wherein the medicament further comprises one or more active agent selected from the group consisting of allogenic plasmatic enzymes, enzymes obtained from tissues, zymogens, and enzyme inhibitors, wherein the allogenic active agent is subjected to a process selected from the group consisting of virus depletion, virus inactivation, and a combination thereof.

126. The method of claim 121, wherein the medicament further comprises one or more active agent selected from the group consisting of antiadherent agents, antiphlogistic agents, antimicrobial agents, cytostatic agents, active substances that are absorbed slowly, and combinations thereof.

127. The method of claim 121, wherein the medicament further comprises one or more allogenic active agents selected from the group consisting of zymogens of the coagulation cascade and enzymes of the coagulation cascade, wherein said active agents have been subjected to a process selected from the group consisting of virus depletion, virus inactivation, and a combination thereof.

128. The method of claim 121, wherein the medicament further comprises an allogenic component selected from the group consisting of particulate cell elements, particulate cell elements applied on virus-safe microspheres, cells, cells applied on virus-safe microspheres, tissues, tissues applied on virus-safe microspheres, and combinations thereof, wherein the microspheres may consist of a substrate selected from the group consisting of allogenic fibrin, allogenic collagen, allogenic collagen plus allogenic fibrin, and combinations thereof.
